(19) United States
(12) Patent Application Publication
(10) Pub. No.: US 2003/0040008 A1
(43) Pub. Date: Feb. 27, 2003

(54) Method for lmmobilizing an analyte on a solid surface

(75) Inventor: Claudia Preininger, Graz (AT)

Correspondence Address: Mark B. Wilson, Fulbright & Jaworski L.L.P., Suite 2400, 600 Congress Avenue, Austin, TX, 78701, US

(73) Assignee: ARC SEIBERSDORF RESEARCH GMBH, Vienna (AT)

(21) Appl. No.: 10/269,395

(22) Filed: Oct. 11, 2002

Related U.S. Application Data
(63) Continuation of application No. PCT/AT01/00108, filed on Apr. 12, 2001.

(30) Foreign Application Priority Data
Apr. 14, 2000 (AT) A 656/2000

(51) Int. Cl. C12Q 1/68; G01N 33/53; G01N 33/542; G01N 33/543; B05D 3/00

(52) U.S. Cl. 435/6; 436/518; 427/2.11; 435/7.9

(57) ABSTRACT

There is disclosed a method for immobilizing an analyte on a solid surface, which is characterized by the following steps: binding a cyclodextrin molecule having at least two functional groups to a solid surface in a manner that at least one functional group of the cyclodextrin molecule can still be covalently bound to an analyte; and

covalently binding the analyte to the surface-bound cyclodextrin molecule.

Alternatively, it is also feasible to covalently bind the analyte to the cyclodextrin molecule first and connect the cyclodextrin molecule with the solid surface subsequently.

The invention relates to a method for immobilizing an analyte on a solid surface as well as conjugates including analytes bound to solid surfaces.

The binding of analytes to solid surfaces is frequently realized using linker molecules connecting the surface with the analyte. Such cross-linkers are above all preferred, if the analyte to be bound to the solid surface is very small, or if an increased free movability of the analyte is desired for the interaction of the analyte with a ligand to be bound to the analyte.

Preferred applications of such analyte/solid-phase conjugates are, on the one hand, purification methods by which ligands to be isolated from complex mixtures can be bound to the immobilized analyte; on the other hand, such conjugates are used in the analytic/diagnostic sector, particularly in the context of screening procedures and, for instance, to detect rare ligands in biologic liquids, or for diagnostic methods in the field of DNA technology. The latter has been using solid phase conjugates as biochips to an ever increasing extent.

Methods for the production of such chips are, for instance, described in WO 98/20967, EP 947 819 A, WO 99/27140 A, DE 19823876 A1, WO 99/57310 A as well as EP 890 651 A1.

The conjugates described in the prior art, however, are either extremely cumbersome and expensive to produce or exhibit unsatisfactory steric properties such as, e.g., a lacking movability of the analytes, an insufficient spacing to the surface of the solid phase (which might lead to undesired electrostatic interactions with the surface) or an arrangement and distribution of the analytes on the solid surface, which is poor to control or cannot be controlled at all.

It is, therefore, the object of the present invention to provide conjugates which have been improved in view of the known prior art and which, in particular, enable simple production involving as few risks as possible while nevertheless providing the analyte in a satisfactory three-dimensional arrangement.

This object is achieved by a method for immobilizing an analyte on a solid surface, which method is characterized by the following steps:

binding a cyclodextrin molecule having at least two functional groups to a solid surface in a manner that at least one functional group of the cyclodextrin molecule can still be covalently bound to an analyte; and

covalently binding the analyte to the surface-bound cyclodextrin molecule.

Alternatively, the immobilization of the analyte on a solid surface can also be accomplished by

covalently binding a cyclodextrin molecule having at least two functional groups to an analyte in a manner that at least one functional group of the cyclodextrin molecule can still be bound to a solid surface; and

binding the cyclodextrin molecule with the bound analyte to a solid surface.

The present invention for the first time makes available analyte solid phase conjugates comprising cyclodextrin linkers. Although cyclodextrins constitute a type of molecule widely used in industrial chemistry for the complexing of a plurality of biomolecules, it has not been possible so far to develop such applications for cyclodextrin molecules because of the lack of cyclodextrin molecules selectively equipped with functional groups. It was only with the introduction of chemically definable cyclodextrin molecules equipped with functional groups (cf. EP 0 697 415 A1) that cyclodextrins could be conjugated to solid phases at all, yet they have continued to serve for the complexing of organic substances.

The conjugates to be produced by the method according to the invention, i.e. conjugates comprising a solid surface, a cyclodextrin bound thereto, and an analyte covalently bound to the cyclodextrin, offer various advantages over the conjugates known from the prior art. Thus, the relatively large cyclodextrin molecule, due to the increased spacer length, provides a largely unlimited free movability of the analyte, which not only decisively enhances the interaction between cross-linker and analyte (and hence facilitates coupling reactions), but also markedly facilitates the interaction with the ligand molecule. Moreover, cyclodextrins are biocompatible, non-toxic and temperature-resistant up to 200° C., thus enabling easy operation without risks and imparting good stability on the conjugate provided according to the invention.

Furthermore, the use of a cyclodextrin as a cross-linker between a solid phase and an analyte provides a high binding capacity, little unspecific adsorption and—for instance, in fluorescence detection—a low (measuring) background which can be further reduced by the selection of suitable solid phases.

Optionally, further cross-linkers may naturally be provided between the cyclodextrin and the solid phase, or cyclodextrin and analyte, e.g. in that a further cross-linker adheres already to the solid surface or in that the analyte has already been modified with a further cross-linker. Examples of such further cross-linkers have been extensively described in the prior art (e.g., dihydrazides, . . . ).

According to the invention, any molecules to be covalently bound to cyclodextrin and, in particular, biomolecules can be used as analytes. According to the invention, preferred analytes encompass nucleic acids, in particular DNA, peptides, proteins, enzymes, in particular oxidoreductases, transferases and hydrolases, antigens, antibodies, receptors, microorganisms (e.g., prokaryotic or eukaryotic cells, viruses, etc.) or mixtures of such analytes.

In a preferred manner, chromatographic materials, metal films (e.g., thin gold films), synthetic surfaces or glass are used as solid surfaces.

Particularly preferred are selectively masked synthetic surfaces and selectively etched glass surfaces, where only parts of the surface are chemically activated and cross-linkers and hence analytes are, thus, provided only on very precisely defined locations on said surfaces. The choice of the respective surface that meets best a particular demand can, however, be readily made by the skilled artisan on grounds of his knowledge or in view of the prior art. Particularly with DNA-chip technology, the carrier materials disclosed in the initially cited patent application are preferably used.

Biochips and, above all, DNA chips are suitable, for instance, for the analysis of pathologically modified gene activity, the elucidation of pathologic mechanisms or the identification of new drug candidates, in the diagnostics and resistance analysis of infectious diseases, but also in the environmental sector for the identification of pathogenic germs.

In the production of chips, DNA carrier molecules are either synthesized in situ on a matrix by the aid of photolithographic techniques using physical masks or are imprinted by various procedures. The manufacture of printed DNA microarrays comprises the steps of activating and coating the solid chip matrix to which biomolecules are fixed through a suitable coupling chemistry.

DNA can be immobilized on carrier material by adsorption, photolithographic deprotection and covalent and ionic binding. Controlled pore glasses (CPG), SiO₂ layers or polymers are used as carrier materials. CPG and SiO₂ surfaces usually are incipiently etched in order to produce on the surface free OH groups which are allowed to react directly with the DNA sequences or can be converted into other functional groups. In the case of polymers as carrier substances, distinction is made between copolymers containing functional groups, polymers into which functional groups can be introduced by chemical modification, chemically inert polymers such as polysulfones or Teflon, which can be activated by radiation (e.g. UV, Co 60), and chemically inert polymers which are covered by functional copolymers.

Examples of polymers already including functional groups whose activation and conversion into other functional groups has been described include polyamide, polyacrylamide and polyester. Unreactive polymers such as, e.g., polyethylene can be grafted with a reactive monomer such as, e.g., glycidylmethacrylate or N-vinylformamide. A very elegant method of introducing functional groups comprises surface modification by plasma treatment. With polypropylene, the inclusion of amino, hydroxy or thiol groups becomes feasible by various plasma treatment sub-types. When using glass as a substrate, object carriers are incipiently etched and amino- or epoxysilanized.

For the production of specific arrays such as, for instance, oligonucleotide arrays, filter materials like nitrocellulose or nylon (Clontech, U.S.A.) with polylysin or glass object carriers derivatized with various silanes, carboxymethylated dextrans (Biacore AB, Sweden) or polyacrylamide gel pads (Packard/Motorola, U.S.A.) are, for instance, used. As opposed to glass surfaces, nylon membranes stand out for their high binding capacities, yet have larger backgrounds than glass in fluorescent detection. Polyacrylamide and dextran are three-dimensional hydrogels exhibiting very high binding capacities and little unspecific binding as in contrast to flat surfaces like glass.

According to the present invention, the choice of the specific cyclodextrin molecule or functional group, as a rule, is not critical, also α-, β or γ-cyclodextrins being applicable, in particular. Preferred reactive groups on the cyclodextrin molecule are selected from halogen, amine, thiol, isothiocyanate and sulfonic acid groups, aromatic groups, preferably aromates with heteroatoms and, in particular, the previously mentioned functional groups, or combinations of the same. A suitable cyclodextrin to be used in the context of the present invention is a monochlorotriazinyl, substituted β-cyclodextrin, which has already been known as a cross-linking agent or surface-modifying agent on textiles or papers, for instance. This β-cyclodextrin derivative is easy to produce, for instance, by treating cyanuric chloride with β-cyclodextrin in water.

Preferably, a cyclodextrin molecule used according to the invention contains 2 to 4 functional groups and, in particular, identical functional groups. In a preferred manner, binding to the solid phase can, thus, also be done covalently. With more than two functional groups per cyclodextrin molecule, also several analytes can be bound to a cyclodextrin molecule.

According to another aspect, the present invention relates to a conjugate comprising a solid surface, a cyclodextrin bound thereto, and an analyte covalently bound to said cyclodextrin. This conjugate is available according to the method of the invention described above.

In a preferred manner, the conjugate according to the invention is configured as a biochip, i.e., the solid surface as well as the analytes are designed according to the known methods established for biochips and incorporated in methods adapted to such biochips, particularly as regards the soft- and hardware detection of reactions occurring on solid surfaces (cf. the already established biochip products by Affimetrix Inc. and Incyte).

Primarily in practical application, the conjugate according to the invention preferably further comprises a ligand molecule specifically bound to the analyte, for instance a complementary nucleic acid, an antibody, an antigen, a receptor ligand, a receptor and the like.

The conjugate according to the invention, above all if the conjugate according to the invention is configured as a biochip, comprises a whole series (library) of analytes, wherein the analyte library is preferably applied on the solid surface in a manner that the localization of different analytes is feasible in a spatially precise manner.

According to a further aspect, the present invention relates to a method for specifically detecting and optionally isolating a ligand molecule from a sample, which method is characterized in that a sample containing the ligand molecule to be detected or isolated (or a sample likely to contain such a ligand molecule) is contacted with a conjugate according to the invention, the ligand molecule is specifically bound to the bound analyte, and this specific bond is verified by measures known per se, whereupon the ligand molecule is optionally separated from the conjugate and isolated. In doing so, the verification of the specific binding can be adapted to the respective ligand/analyte system or accomplished by generally common methods such as (secondary) antibody reactions, dye reactions, signaling methods via the solid phase (biochip), radioactivity or fluorescence labeling, etc.

In the following, the invention will be explained in more detail by way of the following examples as well as the figures of the drawing, to which it is, of course, not limited. Therein:

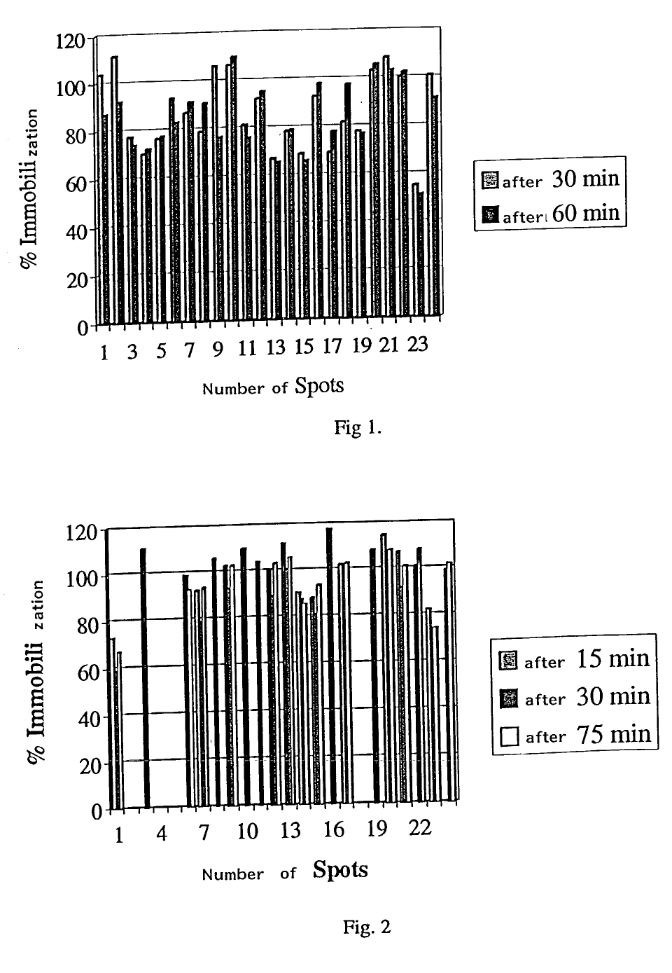

FIG. 1 illustrates the binding of oligonucleotides to PVA/Palam;

FIG. 2 illustrates the binding of oligonucleotides to PVA/Palam/MTC; and

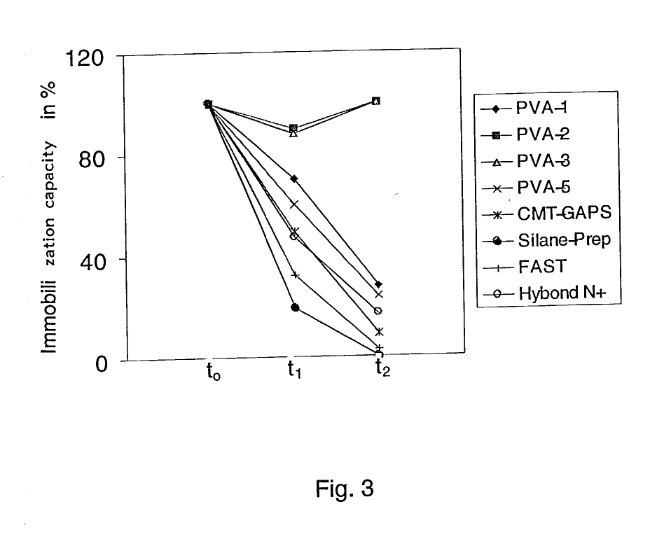

FIG. 3 indicates the immobilization capacity of chips according to the invention in comparison to commercially available products.

### EXAMPLES

Biomolecules like enzymes, antibodies, microorganisms and oligonucleotides (DNA, RNA) can be immobilized by adsorption or embedding in polyvinyl alcohol (PVA). Polyvinyl alcohol has a large, porous surface into which biomolecules can be included. The pore size can be determined by basic or acidic catalysis during gel cross-linking. The results are three-dimensional networks which are mechanically stable and exhibit excellent swelling behaviors in water. PVA gels can be covalently cross-linked by cross-linking with glutaraldehyde, which results in an increased gel hardness. Moreover, functional, reactive groups can be readily introduced into polyvinyl alcohol by acetylation or acylation. PVA with styryl pyridinium groups, for instance, is photosensitive, including biomolecules in its pores upon radiation due to cyclodimerization. PVA gels cross-linked with polyallyl amine and polyacrylic acid are also used for biosensors. On account of the large pores of PVA and its property to swell into a three-dimensional network in water, a high immobilization capacity will be obtained. Yet, such a large-pored matrix also entails the risk of immobilized biomolecules being easily washed out.

1. For the immobilization of biomolecules and, in particular, the immobilization of oligonucleotides on biochips, a thin layer of PVA/polyallyl amine was mounted on an object carrier before the unmodified oligonucleotide (DNA, RNA) was applied on the same. The latter binds electrostatically to the amine via the phosphate group. The bond is consolidated by UV cross-linking.

2. In order to covalently cross-link PVA and covalently bind biomolecules and, in particular, oligonucleotides (DNA/RNA) to PVA, polymers consisting of PVA, polyallyl amine (and polyacrylic acid) and monochlorotriazinyl-β-cyclodextrin, Na-salt (MCT) are prepared. MCT contains 2 to 3 reactive chlorotriazinyl groups per cyclodextrin molecule and binds covalently to polyallyl amine and the amine-modified oligonucleotide. MCT constitutes a bifunctional cross-linker which, on the one hand, is covalently cross-linked to PVA via polyallyl amine and, on the other hand, is able to covalently bind to a bio-molecule comprising nucelophilic groups like —OH and —NH₂. This method offers the following advantages: The PVA gels described are stable, hydrophilic and porous. They swell upon contact with an aqueous solution. As a result, their surfaces will be enlarged and their immobilization capacity improved. The hydrophilic character of the gel provides an easy and rapid access of the biomolecules to the polymer surface, thus reducing unspecific adsorption. Biomolecules in PVA can be immobilized in a solution-like state in a hydrophilic, three-dimensional, porous matrix. On account of the enhanced freedom of movement resulting therefrom, biomolecules immobilized in PVA behave more reactive than those on planar surfaces. Due to the covalent cross-linking of the gel and the covalent binding of the biomolecules to the gel (p. 2), the biomolecules can be prevented from washing out. The applicability of PVA gels, which are suitable not only for the immobilization of oligonucleotides (DNA, RNA), but also for the immobilization of antibodies, enzymes and microorganisms, was demonstrated by way of a 16S rRNA chip. The oligonucleotides which were spotted on PVA/palam or PVA/Palam/MCT, respectively, stayed attached even after washing in a hybridizing solution (20 mM Tris, pH 7.4, 0.01% laurylsulfate, 0.9 M NaCl and 35% formamide). 87% of the oligonucleotide applied on PVA/Palam remained immobilized after excessive washing in a hybridizing solution at 60° C. (FIG. 1).

On PVA/polyallyl amine/MCT, ≧90% of the originally applied oligonucleotide could be immobilized after washing in a hybrid solution at 60° C. (FIG. 2).

### Immobilization of Oligonucleotides on Cross-linked Polyvinyl Alcohol (PVA) for use in DNA Chips

Previously purified microscopic glass platelets were coated by means of a commercially available device (Bickel & Wolf, AT). In doing so, five PVA gels were used (PVA-1 to PVA-5), which contained 5 g of a 10% aqueous PVA (99+% hydrolyzed, MW 85,000-146,000; Aldrich, AT), 0.1 g Palam (Aldrich, AT), 0.1 g monochlorotriazinyl-β-cyclodextrin (β-CD), Cavasol W7 MCT (Wacker, Del.) in 5 ml distilled water and had pH 4 (PVA-1), pH 6.8 (PVA-2), pH 8 (PVA-3) and pH 9 (PVA-4)(upon addition of Na₂CO₃). PVA-5 and PVA-1 were identical except for the addition of β-CD (PVA-5 did not contain β-CD). The thickness of the PVA films was approximately 8 μm (at a resolution of 10 nm). The PVA gels on the chips were polymerized by six freezing (−18° C.) and drying (25° C.) steps. Unmodified and amino-modified EUB338 (5′-GCT GCC TCC CGT AGG AGT-3′), ALFlb (5′-CGT TCG (CT)TC TGA GCC AG-3′) and BET42a (5′-GCC TTC CCA CTT CGT TT-3′) (with and without Cy5 label) were dissolved in 0.05 M phosphate buffer, pH 8, and spotted onto the chips by means of a piezoelectric biochip arrayer. The oligonucleotides were put up in blocks of 5×3 spots of 0.35 to 1 nl. The distance between the spots was 300 μm.

The chips produced according to the invention and containing β-CD were compared with commercially available products such as CMT-GAPS, FAST, Silane-Prep and Hybond N+. The results are indicated in FIG. 3. Therein, to is the fluorescence after spotting; t₁ the fluorescence after blocking; and t₂ the fluorescence after the hybridization of the complementary DNA on the chip. Hence follows that the β-cyclodextrin chips (except for PVA-5) in terms of immobilization capacity clearly outdid all of the commercially available products tested. Immobilization capacities ranging between 85 and 120% after hybridization were markedly better than those of the comparative products (below 40%). The chips according to the invention, thus, exhibit an immobilization capacity largely improved over all other products.

1. A method for immobilizing an analyte on a solid surface, which method is characterized by the following steps:
binding a cyclodextrin molecule having at least two functional groups to a solid surface in a manner that at least one functional group of the cyclodextrin molecule can still be covalently bound to an analyte; and
covalently binding the analyte to the surface-bound cyclodextrin molecule.

2. A method for immobilizing an analyte on a solid surface, which method is characterized by the following-steps:
covalently binding a cyclodextrin molecule having at least two functional groups to an analyte in a manner that at least one functional group of the cyclodextrin molecule can still be bound to a solid surface; and
binding the cyclodextrin molecule with the bound analyte to a solid surface.

3. A method according to claim 1 or 2, characterized in that nucleic acids, in particular DNA, enzymes, in particular oxidoreductases, transferases and hydrolases, antigens, antibodies, receptors, receptor ligands or mixtures of these molecules are used as analytes.

4. A method according to any one of claims 1 to 3, characterized in that chromatographic materials, synthetic surfaces, metal films or glass are used as solid surfaces.

5. A method according to any one of claims 1 to 4, characterized in that a selectively masked synthetic surface is used as a solid surface.

6. A method according to any one of claims 1 to 4, characterized in that a selectively etched glass surface is used as a solid surface.

7. A method according to any one of claims 1 to 6, characterized in that said cyclodextrin is β-cyclodextrin.

8. A method according to any one of claims 1 to 7, characterized in that said cyclodextrin comprises reactive groups selected from halogen, amine, thiol, isothiocyanate and sulfonic acid groups, aromatic groups, preferably aromates with heteroatoms, in particular the previously mentioned functional groups, or combinations thereof.

9. A method according to any one of claims 1 to 8, characterized in that a monochlorotriazinyl-β-cyclodextrin is used as said cyclodextrin.

10. A conjugate comprising a solid surface, a cyclodextrin bound thereto, and an analyte covalently bound to said cyclodextrin.

11. A conjugate according to claim 10, characterized in that it is available according to a method set out in any one of claims 1 to 9.

12. A conjugate according to claim 10 or 11, characterized in that it is configured as a biochip.

13. A conjugate according to any one of claims 10 to 12, characterized in that it further comprises a ligand molecule specifically bound to said analyte.

14. A conjugate according to any one of claims 10 to 13, characterized in that it comprises a library of analytes.

15. A method for specifically detecting and optionally isolating a ligand molecule from a sample, characterized in that a sample containing said ligand molecule is contacted with a conjugate according to any one of claims 10 to 14, the ligand molecule is specifically bound to the bound analyte, and this specific bond is verified by measures known per se, whereupon the ligand molecule is optionally separated from the conjugate and isolated.
